SEQUENCE LISTING (2 sequences)

SEQ ID NO: 1 - protein, 258 aa, synthetic construct
  REGION 1..258: note = cutinase
SNPYQRGPNPTRSALTADGPFSVATYTVSRLSVSGFGGGVIYYPTGTSLTFGGIAMSPGYTADASSLAWLGRRLASHGFVVLVINTNSRFDYPDSRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSTTPKVYVELDNASHFAPNSNNAAISVYTISWMKLWVDNDTRYRQFLCNVNDPALSDFRTNNRHCQ

SEQ ID NO: 2 - protein, 594 aa, synthetic construct
  REGION 1..594: note = MHETase Ideonella
CAGGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRNFATIATDGGHDNAVNDNPDALGTVAFGLDPQARLDMGYNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPPLEHHHHHH

(19) United States
(12) Patent Application Publication
(10) Pub. No.: US 2025/0019513 A1
(43) Pub. Date: Jan. 16, 2025

(54) PROCESS FOR DEGRADING A PLASTIC PRODUCT COMPRISING AT LEAST ONE POLYESTER

(71) Applicant: CARBIOS, CLERMONT-FERRAND (FR)

(72) Inventors: ALAIN MARTY, TOULOUSE (FR); VINCIANE BORSENBERGER, TOULOUSE (FR); VINCENT TOURNIER, TOULOUSE (FR)

(21) Appl. No.: 18/889,745

(22) Filed: Sep. 19, 2024

Related U.S. Application Data
(63) Continuation of application No. 18/560,951, filed on Nov. 15, 2023.

(30) Foreign Application Priority Data
May 20, 2021 (EP) 21174996.5

(51) Int. Cl. C08J 11/10; C08J 3/12; C08J 3/20; C08J 9/00

(52) U.S. Cl. CPC C08J 11/105; C08J 3/12; C08J 3/203; C08J 9/0066; C08J 2367/02

(57) ABSTRACT

The present invention relates to a process for degrading plastic, wherein said plastic products are selected from plastic and/or textiles comprising polyester comprising at least a terephthalic acid monomer. The process of the invention particularly comprises a step of enzymatic depolymerization implemented in acidic conditions at a pH between 3 and 6.

### CROSS-REFERENCE TO RELATED APPLICATIONS

This application is a continuation of U.S. application Ser. No. 18/560,951, filed Nov. 15, 2023, which is the U.S. national stage application of International Patent Application No. PCT/EP2022/063798, filed May 20, 2022.

### REFERENCE TO SEQUENCE LISTING

The Sequence Listing for this application is labeled “Seq-List.xml” which was created on Sep. 3, 2024 and is 4,248 bytes. The entire content of the sequence listing is incorporated herein by reference in its entirety.

### TECHNICAL FIELD

The present invention relates to a process for degrading polyester containing material such as plastic products at an industrial or semi-industrial scale, wherein said plastic products are selected from plastic and/or textiles comprising polyester comprising at least a terephthalic acid monomer. The process of the invention particularly comprises a step of enzymatic depolymerization implemented in acidic conditions at a pH between 3 and 6. The process of the invention is particularly useful for degrading a plastic product comprising polyethylene terephthalate. The invention also relates to a process for producing monomers and/or oligomers from plastic products comprising polyester comprising at least one terephthalic acid monomer.

### BACKGROUND

Plastics are inexpensive and durable materials, which can be used to manufacture a variety of products that find uses in a wide range of applications (food packaging, textiles, etc.). Therefore, the production of plastics has increased dramatically over the last decades. Moreover, most of them are used for single-use disposable applications, such as packaging, agricultural films, disposable consumer items or for short-lived products that are discarded within a year of manufacture. Because of the durability of the polymers involved, substantial quantities of plastics are piling up in landfill sites and in natural habitats worldwide, generating increasing environmental problems. For instance, in recent years, polyethylene terephthalate (PET), an aromatic polyester produced from terephthalic acid and ethylene glycol, has been widely used in the manufacture of several products for human consumption, such as food and beverage packaging (e.g.: bottles, convenience-sized soft drinks, pouches for alimentary items) or textiles, fabrics, rugs, carpets, etc.

Different solutions, from plastic degradation to plastic recycling, have been studied to reduce environmental and economic impacts correlated to the accumulation of plastic waste. Mechanical recycling technology remains the most-used technology, but it faces several drawbacks. Indeed, it requires an extensive and costly sorting and it leads to downgrading applications, due to an overall loss of molecular weight during the process and uncontrolled presence of additives in the recycled products. The current recycling technologies are also expensive. Consequently, recycled plastic products are generally non-competitive compared to virgin plastic.

Recently, innovative processes of enzymatic recycling of plastic products have been developed and described (e.g. WO 2014/079844, WO 2015/097104, WO 2015/173265, WO 2017/198786, WO 2020/094661, and WO 2020/094646). Contrary to traditional recycling technologies, such enzymatic depolymerization processes remove the need for expensive sorting and allow for the recovery of the chemical constituents of the polymer (i.e. monomers and/or oligomers). The resulting monomers/oligomers may be recovered, purified and used to re-manufacture plastic items with equivalent quality to virgin plastic items, so that such processes lead to an infinite recycling of plastics. These processes are particularly useful for recovering terephthalic acid and ethylene glycol from plastic products comprising PET. In these processes, the production of said monomers and/or oligomers, and in particular the production of terephthalic acid, causes a decrease in the pH of the reaction medium which may be detrimental for the degrading enzyme activity. To maintain the pH and thereby an optimum enzyme activity, bases are used massively. However, to recover terephthalic acid by precipitation, a strong acid is used leading to a huge production of salts which are hardly valuable. In addition, use of base and acid as well as the lack of valorisation of the salts significantly impact the cost of these processes.

By working on this issue, the inventors have developed an optimized enzymatic process of degradation of such plastic products, which requires low or no addition of base (and leads to low or no formation of salt) during the process, while maintaining a depolymerization yield satisfactory from economical and industrial point of view.

### SUMMARY OF THE INVENTION

By working on improvements of processes for degrading polyester containing material, such as plastic products, the inventors have discovered that it is possible to implement a depolymerization step under acidic conditions.

It is thus the merit of the inventors to have determined the specific conditions enabling a good balance between base consumption and depolymerization yield acceptable at industrial scale.

In this regard, it is an object of the invention to provide a process for degrading polyester containing material, such as a plastic product, comprising at least one polyester comprising at least a terephthalic acid monomer (TA) wherein the process comprises a step of depolymerization of said at least one polyester performed at a pH between 3 and 6 by contacting said polyester containing material (e.g., plastic product) with at least an enzyme able to degrade said polyester.

It is another object of the invention to provide a process for degrading polyester containing material, such as a plastic product, comprising at least one polyester comprising at least a terephthalic acid monomer (TA) wherein the process comprises an enzymatic depolymerization step performed at a pH regulated between 5 and 5.5, preferably at pH 5.2+/−0.05, by addition of a base in the reaction medium.

It is also an object of the invention to provide a process for degrading polyester containing material, such as a plastic product comprising at least one polyester comprising at least a terephthalic acid monomer (TA) wherein the enzymatic depolymerization step is not regulated and is implemented at a pH between 3 and 4.

### DETAILED DESCRIPTION OF THE INVENTION

### Definitions

In the context of the invention, a “polyester containing material” or “polyester containing product” refers to a product, such as plastic product, comprising at least one polyester in crystalline, semi-crystalline or totally amorphous forms. In a particular embodiment, the polyester containing material refers to any item made from at least one plastic material, such as plastic sheet, tube, rod, profile, shape, film, massive block, fiber, etc., which contains at least one polyester, and possibly other substances or additives, such as plasticizers, mineral or organic fillers. In another particular embodiment, the polyester containing material refers to a plastic compound, or plastic formulation, in a molten or solid state, suitable for making a plastic product. In another particular embodiment, the polyester containing material refers to textile, fabrics or fibers comprising at least one polyester. In another particular embodiment, the polyester containing material refers to plastic waste or fiber waste comprising at least one polyester. Particularly, the polyester containing material is a plastic product.

Within the context of the invention, the terms “plastic article” or “plastic product” are used to refer to any item or product comprising at least one polymer, such as plastic sheet, tube, rod, profile, shape, massive block, fiber, etc. Preferably, the plastic article is a manufactured product, such as rigid or flexible packaging (bottle, trays, cups, etc.), agricultural films, bags and sacks, disposable items or the like, carpet scrap, fabrics, textiles, etc. The plastic article may contain additional substances or additives, such as plasticizers, minerals, organic fillers or dyes. In the context of the invention, the plastic article may comprise a mix of semi-crystalline and/or amorphous polymers and/or additives.

A “polymer” refers to a chemical compound or mixture of compounds whose structure is constituted of multiple repeating units (i.e. “monomers”) linked by covalent chemical bonds. Within the context of the invention, the term “polymer” refers to such chemical compound used in the composition of a plastic product.

The term “polyester” refers to a polymer that contains the ester functional group in their main chain. Ester functional group is characterized by a carbon bound to three other atoms: a single bond to a carbon, a double bond to an oxygen, and a single bond to an oxygen. The singly bound oxygen is bound to another carbon. According to the composition of their main chain, polyesters can be aliphatic, aromatic or semi-aromatic. Polyester can be homopolymer or copolymer. As an example, polyethylene terephthalate is a semi-aromatic copolymer composed of two monomers: terephthalic acid and ethylene glycol.

The term “depolymerization”, in relation to a polymer or plastic article containing a polymer, refers to a process by which the polymer or at least one polymer of said plastic article is depolymerized and/or degraded into smaller molecules, such as monomers and/or oligomers and/or any degradation products.

According to the invention, “oligomers” refer to molecules containing from 2 to about 20 monomer units. As an example, oligomers retrieved from PET include methyl-2-hydroxyethyl terephthalate (MHET) and/or bis (2-hydroxyethyl) terephthalate (BHET) and/or 1-(2-hydroxyethyl) and/or 4-methyl terephthalate (HEMT) and/or dimethyl terephthalate (DMT).

The term “reaction medium” refers to all the elements and compounds (including liquid, enzymes, polyester, monomers and oligomers resulting from the depolymerization of said polyester) present in a reactor during a depolymerization step, also referred as the reactor content.

According to the invention, the “liquid phase of the reaction medium” refers to the reaction medium free of any solid and/or suspended particles. Said liquid phase includes the liquid and all compounds dissolved within (including enzymes, monomers, salts, etc.). This liquid phase can be separated from the solid phase of the reaction medium and retrieved, using means known by one skilled in the art, such as filtration, decantation, centrifugation, etc. In the context of the invention, the liquid phase is notably free of residual polyester (i.e., non-degraded and insoluble polyester) and of precipitated monomers.

### Process of the Invention

By working on the optimisation of enzymatic degrading process of plastic products, the inventors have discovered that it is possible to avoid coproducts (salts) production and to improve the economic return of a plastic product degrading process by reducing the base consumption while maintaining an enzymatic activity compatible with industrial performances. More particularly, the inventors have discovered that an enzymatic depolymerization of polyester may be performed at an acid pH, with low addition of base. Alternatively, said acidic depolymerization step is implemented without any regulation of pH in the reaction medium, i.e. with no addition of base.

Thus, it is an object of the invention to provide a process for degrading a polyester containing material, such as a plastic product, comprising at least one polyester comprising at least a terephthalic acid monomer (TA) wherein the process comprises a step of depolymerization of said at least one polyester performed at a pH between 3 and 6 by contacting the polyester containing material, e.g., the plastic product, with at least an enzyme able to degrade said polyester.

In a preferred embodiment, the enzyme is a depolymerase, more preferably an esterase, even more preferably a lipase or a cutinase.

According to the invention, the enzymatic depolymerization step is implemented at a temperature between 40° C. and 80° C., preferably between 50° C. and 72° C., more preferably between 50° C. and 65° C., even more preferably between 50° C. and 60° C. In an embodiment, the enzymatic depolymerization step is implemented at a temperature between 55° C. and 60° C. or between 50° C. and 55° C. In another embodiment, the enzymatic depolymerization step is implemented between 55° C. and 65° C. In another embodiment, the depolymerization step is implemented between 60° C. and 72° C., preferably between 60° C. and 70° C. In an embodiment, the temperature of the enzymatic depolymerization step is maintained below the Tg of the polyester of interest. Within the context of the invention, the “polyester of interest” refers to the polyester comprising at least a terephthalic acid monomer (TA) targeted by the degradation process. Advantageously, the temperature is maintained at a given temperature+/−1° C.

### Given pH with Regulation

In a particular embodiment, during said depolymerization step, the pH is regulated at a given pH between 3 and 6,+/−0.5, by addition of a base. Any base known by one skilled in the art may be used. Particularly, the pH may be regulated by addition in the reaction medium of a base selected from the group consisting in sodium hydroxide (NaOH), potassium hydroxide (KOH) or ammonia (NH4OH). Advantageously, the base is sodium hydroxide (NaOH). Preferably, the pH is regulated at a given pH+/−0.1, preferably+/−0.05. That is to say that bases are added in the reaction medium in amounts required to prevent any decrease of the pH below said given pH. Particularly, the given pH of the depolymerization step is regulated between 4 and 6, preferably between 5 and 6.

In another embodiment, the given pH is regulated between 4 and 5.5, preferably between 4.5 and 5.5, more preferably between 5 and 5.5 by addition of a base in the reaction medium. Particularly, the given pH is regulated between 5.1 and 5.3, preferably regulated at pH 5.2+/−0.5, preferably+/−0.1, more preferably+/−0.05. Alternatively, the given pH is regulated between 5.3 and 5.5, preferably regulated at pH 5.4+/−0.5, preferably+/−0.1, more preferably +/−0.05. Alternatively, the given pH is regulated between 5.5 and 6.

In an embodiment, the depolymerization step is implemented at a pH regulated between 5.0 and 5.5 and at a temperature comprised between 50° C. and 72° C., preferably between 50° C. and 65° C., more preferably between 50° C. and 60° C. Alternatively, the depolymerization step is implemented at a pH regulated between 5.0 and 5.5 and at a temperature comprised between 65° C. and 72° C. Alternatively, the depolymerization step is implemented at a pH regulated between 5.0 and 5.5 and at a temperature comprised between 60° C. and 65° C.

### Without any Regulation

In another particular embodiment, the pH of the depolymerization step is not regulated, i.e. no base is added in the reaction medium in order to control the pH during the depolymerization step.

Accordingly, the depolymerization step is implemented at a pH between 3 and 5.

Particularly, the depolymerization step is implemented at a pH between 3 and 4, preferably between 3.5 and 4. Alternatively, the depolymerization step is implemented at a pH between 4 and 5, preferably between 4.5 and 5. In an embodiment, the depolymerization step is implemented at a pH between 4.5 and 5 and at a temperature between 50° C. and 60° C. Alternatively, the depolymerization step is implemented at a pH between 4.5 and 5 and at a temperature between 60° C. and 65° C. Alternatively, the depolymerization step is implemented at a pH between 4.5 and 5 and at a temperature between 65° C. and 72° C.

### Enzymes and Microorganisms

According to the invention, the depolymerization step is implemented by contacting the plastic product comprising at least one polyester comprising at least a TA monomer with at least an enzyme able to degrade said polyester. In an embodiment, the depolymerization step is implemented by contacting the plastic product comprising at least one polyester comprising at least a TA monomer with at least a microorganism that expresses and excretes said enzyme able to degrade said polyester.

In an embodiment, said at least one enzyme exhibits a polyester-degrading activity at a pH between 3 and 6 and/or has an optimum pH between 3 and 6. The “optimum pH of an enzyme” refers to the pH at which the enzyme exhibits the highest degradation rate at given conditions of temperature and in a given medium. In another embodiment, said at least one enzyme has an optimum pH between 6 et 10 and still exhibits a polyester-degrading activity at a pH between 3 and 6 and/or at the pH of depolymerization step.

In the context of the invention, a “polyester-degrading activity” can be assessed by any means known by the skilled person. Particularly, a “polyester-degrading activity” can be assessed by measurement of the specific polyester's depolymerization activity rate, the measurement of the rate to degrade a solid polyester compound dispersed in an agar plate, the measurement of the polyester's depolymerization activity rate in reactor, the measurement of the quantity of depolymerization products (EG, TA, MHET, . . . ) released, the mass measurement of the polyester.

In an embodiment, the enzyme is selected from depolymerases, preferably selected from esterases. In a preferred embodiment, the enzyme is selected from lipases or cutinases.

In a particular embodiment, the enzyme is an esterase. Particularly, the esterase is a cutinase, preferably a cutinase coming from a microorganism selected from Thermobifida cellulosityca, Thermobifida halotolerans, Thermobifida fusca, Thermobifida alba, Bacillus subtilis, Fusarium solani pisi, Humicola insolens, Sirococcus conigenus, Pseudomonas mendocina, Thielavia terrestris, Saccharomonospora viridis, Thermomonospora curvata or any functional variant thereof. In another embodiment, the cutinase is selected from a metagenomic library such as LC-Cutinase described in Sulaiman et al., 2012 or the esterase described in EP3517608, or any functional variant thereof including depolymerases listed in WO 2021/005198, WO 2018/011284, WO 2018/011281, WO 2020/021116, WO 2020/021117 or WO 2020/021118. In another particular embodiment, the esterase is a lipase preferably coming from Ideonella sakaiensis or any functional variant thereof including the lipase described in WO 2021/005199. In another particular embodiment, the depolymerase is a cutinase coming from Humicola insolens, such as the one referenced A0A075B5G4 in Uniprot or any functional variant thereof. In another embodiment, the depolymerase is selected from commercial enzymes such as Novozym 51032 or any functional variant thereof.

In another particular embodiment, the enzyme is selected from enzymes having at least 75%, 80%, 85%, 90%, 95%, 96%, 97%, 98%, or 99% identity to the full length amino acid sequence set forth in SEQ ID NO:1, and exhibiting a polyester-degrading activity, particularly a PET-degrading activity.

In an embodiment, the enzyme is selected from enzyme having a PET-degrading activity (PETase) and/or enzyme having a MHET-degrading activity (MHETase).

In the context of the invention, a “MHET-degrading activity” can be assessed by any means known by the skilled person. As an example, the “MHET-degrading activity” can be assessed by measurement of the MHET degradation activity rate by the measurement of the quantity of depolymerization products (EG and TA) released.

In an embodiment, the MHETase may be selected from depolymerases, preferably selected from esterases. In an embodiment, the MHETase is selected from lipases or cutinases. In another embodiment, the MHETase is selected from enzymes belonging to the class EC: 3.1.1.102.

In a particular embodiment, the MHETase is selected from an MHETase isolated or derived from Ideonella sakaiensis, as disclosed in Yoshida et al., 2016, or any functional variant thereof. In another particular embodiment, the MHETase is selected from enzymes having at least 75%, 80%, 85%, 90%, 95%, 96%, 97%, 98%, or 99% identity to the full length amino acid sequence set forth in SEQ ID NO:2.

In a particular embodiment, the PETase and the MHETase are included in a multienzyme system, particularly a two-enzyme system such as the Ideonella sakaiensis PETase/MHETase system disclosed in Knott et al. 2020.

In an embodiment the depolymerization step is implemented by contacting the plastic product comprising at least one polyester with at least two enzymes, preferably with at least two enzymes exhibiting said polyester degrading activity. In a particular embodiment, the plastic product comprises PET and the depolymerization step is implemented by contacting the plastic product comprising at least PET with at least two enzymes, preferably at least one PETase and at least one MHETase. MHETase may be added simultaneously to PETase. Alternatively or in addition, MHETase may be added after PETase, for instance once polyester has been at least partially degraded by PETase. The simultaneous use of a PETase and a MHETase may in particular embodiments lead to a synergistic effect, thus leading to a depolymerization rate higher than the sum of the depolymerization rates obtained with the PETase alone and the MHETase alone.

The enzymes may be in soluble form, or solid phase such as powder form. In particular, they may be bound to cell membranes or lipid vesicles, or to synthetic supports such as glass, plastic, polymers, filter, membranes, e.g., in the form of beads, columns, plates and the like. The enzymes may be in an isolated or purified form. Preferentially, the enzymes of the invention are expressed, derived, secreted, isolated, or purified from microorganisms. The enzymes may be purified by techniques known per se in the art and stored under conventional techniques. The enzymes may be further modified to improve e.g., their stability, activity and/or adsorption on the polymer. For instance, the enzymes are formulated with stabilizing and/or solubilizing components, such as water, glycerol, sorbitol, dextrin, including maltodextrine and/or cyclodextrine, starch, propanediol, salt, etc.

In another embodiment, the depolymerization step is implemented with at least one microorganism that expresses and excretes the depolymerase. In the context of the invention the enzyme may be excreted in the culture medium or towards the cell membrane of the microorganism wherein said enzyme may be anchored. Said microorganism may naturally synthesize the depolymerase, or it may be a recombinant microorganism, wherein a recombinant nucleotide sequence encoding the depolymerase has been inserted, using for example a vector. For example, a nucleotide molecule, encoding the depolymerase of interest is inserted into a vector, e.g. plasmid, recombinant virus, phage, episome, artificial chromosome, and the like. Transformation of the host cell as well as culture conditions suitable for the host are well known to those skilled in the art.

The recombinant microorganisms may be used directly. Alternatively, or in addition, recombinant enzymes may be purified from the culture medium. Any commonly used separation/purification means, such as salting-out, heat shock, gel filtration, hydrophobic interaction chromatography, affinity chromatography or ion exchange chromatography may be used for this purpose. In particular embodiments, microorganisms known to synthesize and excrete depolymerases of interest may be used.

According to the invention, several enzymes and/or several microorganisms may be used together or sequentially during the depolymerization step.

According to the invention, the enzyme quantity in the reaction medium is comprised between 0.1 mg/g and 15 mg/g of the targeted polyester, preferably comprised between 0.1 mg/g and 10 mg/g, more preferably comprised between 0.1 mg/g and 5 mg/g, even more preferably between 0.5 mg/g and 4 mg/g. Preferably, the enzyme quantity in the reaction medium is at most 4 mg/g, preferably at most 3 mg/g, more preferably at most 2 mg/g of the targeted polyester. When at least one PETase and at least one MHETase are used, the amount of PETase in the reaction medium is comprised between 0.1 and 10 mg/g of the targeted polyester, preferably between 0.1 mg/g and 5 mg/g, more preferably between 0.5 mg/g and 4 mg/g and the amount of MHETase in the reaction medium is comprised between 0.1 and 5 mg/g of the targeted polyester, preferably between 0.1 mg/g and 2 mg/g.

According to the invention, during the depolymerization step, additional amounts of enzymes (such as PETase and/or MHETase), may be added to the reaction medium, continuously or sequentially. Particularly, additional amounts of MHETase may be added, once or several times during the depolymerisation step.

In an embodiment, the depolymerization step is implemented by contacting simultaneously the plastic product with at least one PETase and at least one MHETase, the pH of the depolymerization step being regulated between 5.0 and 5.5, and the temperature being maintained between 50° C. and 72° C., preferably between 50° C. and 65° C., more preferably between 50° C. and 60° C. Alternatively, the depolymerization step is implemented at a temperature comprised between 65° C. and 72° C. or at a temperature comprised between 60° C. and 65° C. Optionally, additional amounts of enzymes (PETase and/or MHETase) may be added once or several times to the reaction medium during the depolymerization step.

In an embodiment, the depolymerization step is implemented by contacting simultaneously the plastic product with at least one PETase and at least one MHETase, the pH of the depolymerization step being regulated at pH 5.2+/−0.05, and the temperature regulated between 50° C. and 65° C.+/−1° C. Optionally, additional amounts of enzymes (PETase and/or MHETase) may be added once or several times to the reaction medium during the depolymerization step.

In an embodiment, the depolymerization step is implemented by contacting simultaneously the plastic product with at least one PETase and at least one MHETase, the pH of the depolymerization step being regulated at pH 5.2+/−0.05, and the temperature regulated at 54° C.,+/−1° C. Optionally, additional amounts of enzymes (PETase and/or MHETase) may be added once or several times to the reaction medium during the depolymerization step.

In another embodiment, the depolymerization step is implemented by contacting the plastic product with at least one PETase, the pH being regulated at pH 5.2+/−0.05 and the temperature regulated at 54° C.,+/−1° C. Additional amounts of MHETase are further added once or several times to the reaction medium during the depolymerization step. For instance, MHETase is added once PETase has depolymerized at least part of the polyester into oligomers. Advantageously, the PETase is selected from enzymes having at least 75%, 80%, 85%, 90%, 95%, 96%, 97%, 98%, or 99% identity to the full length amino acid sequence set forth in SEQ

ID NO: 1, and exhibiting a polyester-degrading activity and the MHETase is selected from enzymes having at least 75%, 80%, 85%, 90%, 95%, 96%, 97%, 98%, or 99% identity to the full length amino acid sequence set forth in SEQ ID NO:2.

### Polyesters

In an embodiment, the process of the invention is implemented with plastic products from plastic waste collection and/or post-industrial waste. More particularly, the process of the invention may be used for degrading domestic plastic wastes, including plastic bottles, plastic trays, plastic bags, plastic packaging, soft plastics and/or hard plastics, even polluted with food residues, surfactants, etc. Alternatively, or in addition, the process of the invention may be used for degrading used plastic fibers, such as fibers providing from fabrics, textiles and/or and industrial wastes. More particularly, the process of the invention may be used with PET plastic and/or PET fiber waste, such as PET fibers coming from fabrics, textile, and/or tires.

According to the invention, the plastic product comprises at least one polyester selected from polyethylene terephthalate (PET), polytrimethylene terephthalate (PTT), polybutylene terephthalate (PBT), polyethylene isosorbide terephthalate (PEIT), polybutylene adipate terephthalate (PBAT), polycyclohexylenedimethylene terephthalate (PCT), glycosylated polyethylene terephthalate (PETG), poly (butylene succinate-co-terephtalate) (PBST), poly (butylene succinate/terephthalate/isophthalate)-co-(lactate) (PBSTIL) and blends/mixtures of these polymers, preferably selected from polyethylene terephthalate (PET).

In an embodiment, the plastic product comprises at least one amorphous polyester targeted by the degrading process.

In an embodiment, the plastic product comprises at least one crystalline polyester and/or at least one semi-crystalline polyester targeted by the degrading process. In the context of the invention, “semi-crystalline polyester” refers to partially crystalline polyester wherein crystalline regions and amorphous regions coexist. The degree of crystallinity of a semi-crystalline polyester may be estimated by different analytical methods and typically ranges from 10 to 90%. For instance, Differential Scanning calorimetry (DSC) or X-Ray diffraction may be used for determining the degree of crystallinity of polymers.

In an embodiment, the plastic product comprises crystalline polyester and/or semi-crystalline polyester, and amorphous polyester, targeted by the degrading process.

In an embodiment, the plastic article may be pretreated prior to the depolymerization step in order to physically change its structure, so as to increase the surface of contact between the polyester and the enzymes and/or to decrease the microbial charge coming from wastes. Examples of pretreatments are described in the patent application WO 2015/173265.

According to the invention, it is possible to submit the polyester of the plastic product to an amorphization step prior to the depolymerization step by any means known by one skilled in the art. An example of amorphization process is described in the patent application WO 2017/198786. In a particular embodiment, the polyester is submitted to an amorphization process followed by a granulation and/or micronization process prior to the depolymerization step.

Alternatively, it is possible to submit the plastic article to a foaming step prior to the depolymerization step by any means known by one skilled in the art. An example of foaming pretreatment process is described in the patent application PCT/EP2020/087209.

In a preferred embodiment, the plastic product is pretreated prior to the depolymerization step and the polyester of interest of the plastic product exhibits a degree of crystallinity below 30% before being submitted to the depolymerization step, preferably a degree of crystallinity below 25%, more preferably below 20%.

### Reactor

According to the invention, the process may be implemented in any reactor having a volume greater than 500mL, greater than 1 L, preferably greater than 2 L, 5 L or 10 L. In a particular embodiment, the process is implemented at a semi-industrial or an industrial scale.

Accordingly, the process may be implemented in a reactor having a volume greater than 100 L, 150 L, 1 000 L, 10 000 L, 100 000 L, 400 000 L.

In the context of the invention, the total volume of the reactor is advantageously at least 10% greater than the volume of the reaction medium, or reactor content.

According to the invention, the initial reaction medium comprises at least a plastic product comprising at least one polyester comprising at least terephthalic acid monomer, a liquid, and at least one enzyme able to degrade said polyester.

In a preferred embodiment, the reaction medium comprises as a liquid an aqueous solvent such as buffer and/or water, preferably water. In a preferred embodiment, the liquid in the reaction medium is free of non-aqueous solvent, especially inorganic solvent. In a particular embodiment, the liquid in the reaction medium consists in water only.

According to the invention, the reactor content is maintained under agitation during the process. The speed of the agitation is regulated by one skilled in the art so as to be sufficient to allow the suspension of the plastic product in the reactor, the homogeneity of the temperature and the precision of the pH regulation if any.

In an embodiment, the concentration of polyester introduced before the depolymerization step is above 150 g/kg in relation to the total weight of the initial reaction medium, preferably above 200 g/kg, more preferably above 300 g/kg, even more preferably above 400 g/kg.

In a particular embodiment, the concentration of polyester introduced before the depolymerization step is comprised between 200 g/kg and 400 g/kg, preferably between 300 g/kg and 400 g/kg. Alternatively, the concentration of polyester introduced before the depolymerization step is comprised between 400 g/kg and 600 g/kg.

In an embodiment, during the depolymerization step, additional polyester and/or enzymes may be added in the reaction medium, continuously or sequentially.

Particularly, polyester may be added in order to reach a final concentration of polyester introduced in the reaction medium comprised between 300 g/kg and 600 g/kg of polyester, preferably between 400 g/kg and 600 g/kg, more preferably between 500 g/kg and 600 g/kg. The final concentration of polyester corresponds to the total quantity of polyester introduced during the whole degrading process in the reaction medium based on the total weight of the reaction medium before the depolymerization step.

In an embodiment, the concentration of polyester introduced before the depolymerization step is below 300 g/kg in relation to the total weight of the reaction medium, preferably between 200 g/kg and 300 g/kg, and further polyesters are added during the depolymerization step in order to reach a final concentration of polyester introduced in the reaction medium above 400 g/kg, more preferably above 500 g/kg, even more preferably between 500 g/kg and 600 g/kg. Optionally, further enzymes are also added during the depolymerization step.

### Purification

In a particular embodiment, the process for degrading polyester containing material (e.g., a plastic product) further comprises a step of recovering and optionally purifying the monomers and/or oligomers and/or degradation products, preferably terephthalic acid, resulting from the step(s) of depolymerization. Monomers and/or oligomers and/or degradation products resulting from the depolymerization may be recovered, sequentially or continuously. A single type of monomers and/or oligomers or several different types of monomers and/or oligomers may be recovered. The recovered monomers and/or oligomers and/or degradation products may be purified, using all suitable purifying method and optionally conditioned in a re-polymerizable form. An example of purification is described in the patent application WO 1999/023055. In a particular embodiment, the recovery of TA under solid form comprises separating the solid phase from the liquid phase of the reaction medium by filtration.

The solid phase recovered may be dissolved and/or dispersed in a solvent selected from water, DMF, NMP, DMSO, DMAC or any solvent known to solubilized TA and filtered to remove impurities. Solubilized TA can then be recrystallized by any means known by one skilled in the art.

In an embodiment, after the depolymerization step, a MHETase is added in the reaction medium before the purification process, in order to hydrolyze the MHET produced during the depolymerization step(s) to produce TA.

In a preferred embodiment, the repolymerizable monomers and/or oligomers may then be reused to synthesize polymers. One skilled in the art may easily adapt the process parameters to the monomers/oligomers and the polymers to synthesize.

Accordingly, it is also an object of the invention to provide a process for recycling a polyester containing material, such as a plastic article, comprising at least one polyester comprising at least one TA monomer, preferably PET, and/or to provide a method of producing monomers and/or oligomers and/or degradation products, preferably TA, from a plastic article comprising at least one polyester having at least one TA monomer, comprising submitting the plastic article to an enzymatic depolymerization step performed at a pH between 3 and 6, and recovering and optionally purifying the monomers and/or oligomers.

All particular embodiments exposed above in connection with the process for degrading polyester containing material, such as plastic product, also apply to the methods of producing monomers and/or oligomers and to the methods of recycling.

### EXAMPLES

### Example 1—Process of Degrading a Plastic Product Comprising PET Comprising an Enzymatic Depolymerization Step Regulated at pH 5.20+/−0.05

Washed and colored flakes from bottle waste comprising 98% of PET with a mean value of crystallinity of 27% were foamed, by submitting the flakes (98.5% by weight based on the total weight of the mix introduced in the extruder) to an extrusion with 1% by weight of citric acid (Orgather exp 141/183 from Adeka) and 0.5% by weight of water, based on the total weight of the mix introduced in the extruder, in a twin-screw extruder Leistritz ZSE 18 MAXX at a temperature above 250° C. The resulting extrudate was granulated into 2-3 mm solid pellets with a crystallinity level of 7% (i.e., foamed PET).

The degrading process of the invention was carried out in 500 mL reactors using a variant of LC-Cutinase (Sulaiman et al., Appl Environ Microbiol. 2012 March). Such variant (herein after “LCC-ICCIG”) corresponds to the enzyme of SEQ ID NO: 1 with the following mutations F208I+D203C+S248C+V1701+Y92G, as compared to SEQ ID NO: 1, and was expressed as recombinant protein in Trichoderma reesei.

At the beginning of the process, foamed PET was added in the reactor at a concentration of 200 g/kg based on the total weight of the initial reaction medium and LCC-ICCIG was added at 4 mg/g PET in 100 mM phosphate buffer pH8. During the depolymerization step, the temperature was regulated at 56° C. and the pH of the reaction medium was regulated at pH 5.2±0.05 by addition of 5% NaOH solution.

The PET depolymerization rate was measured via regular sampling. Samples from the reaction medium were analyzed by Ultra High Performance Liquid Chromatography (UHPLC) for measuring the amount of equivalent terephthalic acid produced.

The samples were diluted in 100 mM potassium phosphate buffer, pH 8. One mL of samples or diluted samples were mixed with 1 mL of methanol and 100 μL of 6 N HCl. After homogenization and filtration through a 0.45 um syringe filter, 20 μL of sample were injected into the UHPLC, Ultimate 3000 UHPLC system (Thermo Fisher Scientific, Waltham, MA) including a pump module, a sampler automatic, a column thermostated at 25° C. and a UV detector at 240 nm. The molecule of terephthalic acid and oligomers (MHET and BHET) were separated using a gradient of methanol (30% to 90%) in 1 mM H2SO4 at 1 m/min through a HPLC Discovery HS C18 column (150 mm×4.6 mm, 5 μm) equipped with a precolumn

(Supelco, Bellefonte, PA). TA alone, MHET and BHET were measured according to standard curves prepared from commercially available TA and BHET and internally synthesized MHET (by partial base-catalyzed hydrolysis of BHET). The TA equivalent is the sum of the measured TA, MHET and BHET.

The depolymerization rate after 140 h of reaction was 38%. After 140 h of reaction, the theoretical base consumption (Y base) was determined and corresponds to the base quantity added in the reaction medium in order to solubilize the precipitated TA (or to the base quantity that should have been introduced if the whole process would have been implemented at pH 8 with the same enzyme). The base consumption saving (in %) during said process was then determined by the following formula:

$$base consumption saving = 100 - \frac{total base consumption \times 100}{Y base}$$

The results show that the process of the invention at pH 5.2 allows a base economy of 25% as compared to a base regulated process at pH8.

### Example 2: Process of Degrading a Plastic Product Comprising PET Comprising an Enzymatic Depolymerization Step Regulated at pH 5.20+/−0.05 with Addition of a MHETase

The process was implemented with same foamed PET flakes as described in Example 1. The same variant (“LCC-ICCIG”) that corresponds to the enzyme of SEQ ID NO: 1 with the following mutations F208I+D203C+S248C+V1701+Y92G were used. However, in this case the enzyme was expressed as recombinant protein in Bacillus subtilis

At the beginning of the process, foamed PET flakes were added in the reactor at a concentration of 200 g/kg based on the total weight of the initial reaction medium and LCC-ICCIG was added at 4 mg/g PET in 300 mM acetate sodium buffer pH 5.2 as well as 6.5 mg of Ideonella sakaiensis MHETase of SEQ ID NO:2. During the depolymerization step, the temperature was regulated at 54° C. and the pH of the reaction medium was regulated at pH 5.2±0.05 by addition of 25% NaOH solution.

Additional amounts of MHETase were added according to Table 1 below.

TABLE 1
Addition of MHETase in the reactor

| | | |
|---|---|---|
| Reaction time (h) | 24 | 52 |
| Quantity of MHETase added (mg) | 6.5 | 5.2 |

One control (Control-1) wherein the depolymerization was implemented in absence of MHETase was also performed.

The depolymerization rate after 71 h and the base consumption saving compared to a regulated process at pH 8 with addition of MHETase were of 58% and 48.4%, respectively.

The depolymerization rate after 71 h and the base consumption saving compared to a regulated process at pH 8 of Control-1 (i.e., without addition of MHETase) were of 46.1% and 39.3%, respectively.

These results show that adding MHETase allows to further increase the depolymerization rate of the reaction when performed at acid pH.

1. A process for producing oligomers from a plastic product comprising PET, comprising submitting the plastic product to an enzymatic depolymerization step performed at a pH between 3 and 6.5 by contacting the plastic product in the reaction medium with at least one enzyme having a PET-degrading activity (PETase), and recovering the oligomers, wherein the oligomers are MHET.

2. The process according to claim 1, wherein the enzyme having a PET-degrading activity is an esterase, a lipase or a cutinase.

3. The process according to claim 1, wherein the pH of the depolymerization step is regulated between 3 and 6 by addition of a base in the reaction medium.

4. The process according to claim 3, wherein the base is selected from the group consisting of sodium hydroxide (NaOH), potassium hydroxide (KOH) and ammonia (NH4OH).

5. The process according to claim 1, wherein the process is implemented at a temperature comprised between 50° C. and 72° C.

6. The process according to claim 1, wherein the concentration of PET introduced in the reaction medium before the depolymerization step is above 150 g/kg based on the total weight of the reaction medium.

7. The process according to claim 1, wherein said plastic product comprising PET is submitted to an amorphization and/or a foaming step prior to the depolymerization step.

8. The process according to claim 1, wherein the process further comprises a step of recovering and optionally purifying the MHET and/or monomers resulting from the depolymerization of said polyester.

9. The process according to claim 8, wherein said method comprises recovering and purifying the MHET and/or monomers resulting from the depolymerization of said polyester using solvents selected from water, DMF, NMP, DMSO, or DMAC.
